(19) United States
(12) Patent Application Publication
(10) Pub. No.: US 2019/0099267 A1
(43) Pub. Date: Apr. 4, 2019

(54) REPLACEMENT CARDIAC VALVE

(71) Applicant: MVALVE TECHNOLOGIES LTD., Herzliya (IL)

(72) Inventors: Amit TUBISHEVITZ, Tel Aviv (IL); Shay DUBI, Tel Aviv (IL); Assif STOFFMAN, Beit Yitzhak - Sha'ar Hefer (IL)

(21) Appl. No.: 16/087,220

(22) PCT Filed: Mar. 22, 2017
(86) PCT No.: PCT/IL2017/050360

§ 371 Date: Sep. 21, 2018

Related U.S. Application Data
(60) Provisional application No. 62/311,421, filed on Mar. 22, 2016.

(51) Int. Cl. A61F 2/24

(52) U.S. Cl. CPC A61F 2/2418; A61F 2/2427; A61F 2250/0069; A61F 2230/0065; A61F 2210/0014; A61F 2230/0093; A61F 2220/0008; A61F 2250/006; A61F 2230/0069; A61F 2/2454; A61F 2/2409; A61F 2220/0075

(57) ABSTRACT

The present invention is directed to a prosthetic atrioventricular valve (10), suitable for transvascular delivery to a heart valve annulus, comprising a tubular stent-like portion (14) that is continuous in its upper part with a laterally expanded crown-like portion (12), wherein at least said stent-like portion is covered with a covering layer (32) comprising a biocompatible fabric, pericardial tissue or combination thereof, and wherein said stent-like portion is fitted with two or more valve leaflets (36a, 36b). The invention also includes within its scope a cardiac valve support device (18) comprising a tubular stent-like portion that is continuous in its upper part with a laterally expanded crown-like portion.

### FIELD OF THE INVENTION

The present invention relates to an intracardiac device for implantation at a cardiac atrioventricular valve annulus. More specifically, the present invention provides a replacement mitral valve, as well as a mitral valve support, both of which are crimpable to a very small diameter, thereby enabling safe transseptal delivery.

Heart valve regurgitation occurs when the heart leaflets do not completely close when the heart contracts. When the heart contracts, blood flows back through the improperly closed leaflets. For example, in mitral valve regurgitation blood flows back through the mitral valve and into the left atrium when the ventricle contracts. Regurgitation may occur due to pathological involvement of the valve leaflets themselves (primary regurgitation) or following dilatation of the ventricle, such that there is a spreading apart of the valve leaflets, thereby leading to poor coaptation (secondary regurgitation).

In some cases, regurgitation may be corrected by attempting to remodel the native leaflets, such as with surgical correction, clips, sutures, hooks, etc., to allow them to close completely with good coaptation when the heart contracts. However, in other cases, particularly when the condition is more advanced, the entire valve needs to be replaced with a prosthesis, either mechanical or biological.

Until relatively recently, the implantation of prosthetic cardiac valves involved an open-heart procedure and the need to stop the heart and connect the patient to a heart machine. However, in common with many other surgical procedures, various attempts have been made to develop prosthetic cardiac valves that may be delivered using a non-invasive or minimally invasive approach, ideally, while the heart is still beating.

Although catheter-based aortic valve delivery and implantation has met with a reasonable degree of success, the use of such a minimally invasive approach for mitral valve replacement has proven to be much more problematic.

One particular problem is related to the fact that the mitral valve annulus has a non-uniform anatomy, and is much larger than the aortic valve annulus. It has been estimated that the mitral valve annulus is about 2.4 cm to about 5 cm in diameter, while the aortic valve annulus is a generally tubular structure, estimated to be about 1.6 cm to about 2.5 cm in diameter.

Consequently, replacement mitral valves are correspondingly larger than those prosthetic valves intended for use at the aortic valve annulus. Although some attempts have been made to deliver and implant a one-piece replacement mitral valve, it has proven very difficult to provide a device that can be collapsed down to have a sufficiently small delivery profile to enable delivery via vascular catheters. The situation is further complicated by the seemingly contradictory requirements of (i) being collapsible (or crimpable) to a very small diameter, and (ii) possessing anchoring mechanisms that are sufficiently robust for the device to be mechanically stable over long term use in the contracting heart. Indeed, many of the prior art solutions to increasing the stability of implanted prosthetic mitral valves have involved 360-degree stabilization to the annulus and/or native valve leaflets. The anchoring structures required are often too bulky (or otherwise difficult to crimp), such that nearly all of the prior art replacement mitral valves intended for minimally-invasive delivery are incapable of being crimped to a diameter of less than about 30 French. While delivery systems of this diameter may be used in a transapical approach (following an incision in the chest wall), these large-diameter crimped devices are not small enough to facilitate their safe, atraumatic delivery via the vasculature in a transvascular, transseptal approach, for example in the same delivery profile of the Mitraclip (Abbott vascular) which is delivered transseptally for mitral valve repair and crimps to a diameter of 24 Fr.

A further technical problem encountered during the development of crimpable replacement heart valves is the paravalvular leakage that commonly occurs, unless potentially bulk-increasing sealing elements are added to the device. Such elements clearly increase the minimal crimp diameter that may be achieved, and thus limit the transvascular use of those devices.

The present invention addresses these problems and provides intracardiac devices suitable for implantation at the mitral valve annulus that (a) are crimpable to a very small diameter, suitable for delivery in a flexible vascular catheter, (b) have a high level of mechanical stability within the atrioventricular annulus and (c) are constructed such that paravalvular leakage is either entirely limited or reduced to insignificant levels.

### SUMMARY OF THE INVENTION

In one aspect, the present invention is directed to a prosthetic atrioventricular valve suitable in size, shape and construction for transvascular delivery and implantation at an atrioventricular annulus, comprising a tubular stent-like portion that is continuous, in its upper part with a laterally expanded crown-like portion, wherein said stent-like portion is fitted with two or more valve leaflets.

For the purpose of the present disclosure, the term “tubular stent-like portion” (and similar forms of this phrase) refers to the portion of the intracardiac device which is constructed from a radially expandable tube comprising open mesh-like cells. In other words, this portion has the same construction as many or most of the stent devices known to the skilled artisan in this field. The tubular stent-like portion has the external form of a short hollow tube, which in use, is implanted such that its long axis is aligned along the longitudinal axis of the heart. This tubular structure has a lower free edge (to which may be connected other structures, as will be described hereinbelow) and upper edge that is continuous with the expanded crown-like portion of the device. This latter portion similar has an open meshwork construction and in its medial region (i.e. close to the upper edge of the tubular stent-like portion) is disposed in a generally horizontal direction (i.e. at approximately right angles to the longitudinal axis of said tubular stent-like portion). In most embodiments, the more lateral region of the lateral crown structure curves upwards and outwards, such that it has an outline shape similar to that of the lower portion of the atrial wall of the heart, immediately above the atrioventricular annulus.

The stent-like portion of the prosthetic valve (also referred to hereinbelow as “the device”) provides support for the valve leaflets that are attached to its lower portion. The function of the aforementioned laterally expanded crown portion is to cover part of the atrial wall in the region of the annulus, thereby both increasing the mechanical stability of the device and improving the fluid sealing of the device against the cardiac tissues, and thus preventing or reducing paravalvular leakage.

The device of the present invention may further comprise one or more (preferably two or more, most preferably only two) anchoring elements. Preferably, said anchoring elements are elongate anchoring arms (also referred to herein as anchoring wings), each having a free end, which in use is positioned against the ventricular wall, and a root that arises from the stent-like portion of the device. The anchoring elements function to maintain the device in position after its deployment in the heart, preventing migration of the device during cardiac contraction. In one preferred embodiment, the device of the present invention comprises only two elongate anchoring arms, mutually separated by approximately 180 degrees. Further details of suitable anchoring arms for use as part of the present invention are contained in a co-owned US patent application which was published with publication number US 2014/0200662, and whose contents are incorporated herein by reference.

The present device is covered externally, and optionally on its internal side as well, by a covering layer. In one preferred embodiment, the covering layer is a biocompatible fabric or biocompatible polymer cover (for example, Dacron, PTFE, polyester etc.—referred to hereinbelow as “fabric”).

In another preferred embodiment, the covering layer comprises a biological tissue, preferably pericardial tissue. In some cases, both non-biological and pericardial tissue are used to construct the covering layer.

In prior art intracardiac devices of various types, fabric covers are used both to conceal potentially traumatic metal edges and to increase fluid sealing around the cardiac tissues. While this is also the case in the present invention, the fabric cover also makes a significant contribution to the mechanical properties of the prosthetic valve device, by means of fixing an outer limit on the radial expansion of the stent-like portion. In the absence of such a cover (whether made of a biocompatible fabric or a biological material such as pericardial tissue), the device of the present invention would not be able to achieve the required level of mechanical stability within the atrioventricular valve annulus. The reason for this is that the supra-annular and infra-annular portions of said stent-like portion would be able to undergo further, undesirable elastic expansion and expand in a radially outward direction as a result of the mechanical forces generated by the contracting heart and associated movement of blood. This lack of rigidity would significantly reduce the mechanical stability of the valve, and would be expected to contribute to increased paravalvular leakage and possible movement and ultimate failure of the implant. Consequently, the fabric or tissue cover around (and/or inside) the device—particularly in its stent-like region—is a key inventive feature of the present invention.

In a preferred embodiment of the present invention, the device may also comprise a lower fabric (or pericardial tissue) sleeve, having an essentially tubular form, attached at its upper margin to the stent-like portion (and/or continuous with the cover attached to said portion), having a lower free margin. Preferably, but not obligatorily, this lower sleeve is supported by (and thus attached to) a metallic support frame that is attached superiorly to the inferior portion of the stent-like region of the device. This lower sleeve support frame may be constructed in a number of different forms, as will be described hereinbelow.

The two or more aforementioned valve leaflets are attached to the stent-like portion, for example they may be attached at their upper margins to the lower border of the stent-like portion, or they may be attached to the upper border of the stent-like portion. The said “attachment” comprises suturing the valve leaflets to the metal frame. In one preferred embodiment, the device comprises two leaflets only, while in other preferred embodiments, the device comprises three leaflets only.

Devices fitted with more than three numbers of leaflets are also encompassed with the scope of the present invention.

In some particularly preferred embodiments, the replacement valve device further comprises one or more metallic commissural struts, each of which may have a free latero-inferior end and an upper end arising from (and continuous with) the stent-like portion of the device, in the region close to the root portion of the anchoring arms. Said free end of each commissural strut is located at the junction between two adjacent fabric (or pericardial) valve leaflets, and provides mechanical support for the fabric in this region, thereby preventing the collapse and improving the function of said valve leaflets. In view of the above-described location of the commissural struts, the number of said struts is generally equivalent to the number of leaflets present in the device.

In other embodiments, the commissural struts may arise from the upper part or from the mid part of the stent-like portion.

In a highly-preferred embodiment of the present invention, the device is suitable in size and shape for implantation at the mitral valve annulus in the heart of a human subject in need of mitral valve replacement.

In another aspect, the present invention is directed to a cardiac valve support device suitable in size, shape and construction for transvascular delivery and implantation at an atrioventricular annulus, comprising a tubular stent-like portion that is continuous, in its upper part with a laterally expanded crown-like portion, wherein at least said stent-like portion is covered with a covering layer comprising a material selected from the group consisting of a biocompatible fabric, pericardial tissue and a combination of both a fabric and pericardial tissue. The valve support device of the present invention may optionally further comprise the anchoring elements, covering layer(s), commissural struts and/or lower fabric or pericardial sleeve as disclosed hereinabove in connection with the prosthetic atrioventricular valve of the present invention.

The valve support device of this aspect of the present invention is suitable for use in a two-step atrioventricular valve replacement method. In a method of this type, said valve support device is initially delivered to the atrioventricular annulus to be treated (e.g. the mitral valve annulus). In a preferred embodiment of the valve support device of the present invention, said device comprises (as described hereinabove) only two elongate anchoring elements which are mutually separated around the circumference of the generally circular device by about 180 degrees. Upon implantation within the valve annulus, the rotational positioning of the device is adjusted such that the two anchoring elements become located at either end of the commissural line of the native valve. As a consequence of this arrangement, the native valve leaflets are able to continue to open and close throughout and after the first step of the two-step method. Subsequently, a prosthetic cardiac valve (such as commercially available aortic valves including, but not limited to, the following valves: Sapien Valve (Edwards Lifesciences Inc., US), Lotus Valve (Boston Scientific Inc., US), CoreValve (Medtronic Inc.) and DFM valve (Direct Flow Medical Inc., US) is delivered to the cardiac valve being replaced. Said valve is then caused or allowed to expand from its collapsed delivery configuration to its expanded working configuration within the central cavity of the previously implanted valve support device. From this point onwards, the valve leaflets of the prosthetic valve take over the task previously performed by the native leaflets, which have now been displaced laterally by the implanted prosthetic valve.

Further technical details concerning two-step mitral valve replacement methods may be obtained from the co-owned international patent applications published as WO 2013/128436 and WO 2012/031141, the contents of which are incorporated herein in their entirety.

It will thus be appreciated that the main difference between the currently-disclosed prosthetic valve and the currently-disclosed valve support device is that the latter device lacks the valve leaflets (and optionally, also the leaflet support structures) that are an obligatory feature of the former device.

One additional difference between the prosthetic valve of the present invention and the valve support device disclosed herein relates to the internal diameter of the tubular stent-like portion of each of these devices. Thus, in the case of the prosthetic valve embodiment, the internal diameter of the tubular stent-like portion (when the device is in its expanded, uncrimped conformation) is in the range of 20-35 mm. In the case of the valve support device embodiment, the comparable diameter is in the range of 19-32 mm. In both cases, the lateral crown-like has a diameter which is greater than that of the tubular stent-like portion, and generally in the range of about 35-65 mm.

In a further aspect, the present invention is also directed to an intracardiac device comprising a hollow plug element for improving sealing of the device within the native cardiac valve annulus. In its most general form, said plug element has an outline form (i.e. when viewed from above or below) similar to, or the same as, the cardiac valve annulus into which the intracardiac device is to be implanted. Thus, in many cases (such when incorporated into a valve support device or prosthetic valve as disclosed and described herein), the outline shape of the plug element is generally circular. Again, in its most general form, the plug element may be defined as having an upper perimeter and a lower perimeter, with tapering sides connecting said perimeters, such that the external diameter of said plug element is larger at its upper face than at its lower face. It is to be noted, in this regard, that the terms “upper” and “lower” in this context refer to the extremities or perimeters of the plug element that are closest, respectively, to the laterally expanded crown-like portion, and to the tubular stent-like portion. In other words, following implantation of the device within a cardiac annulus, the upper perimeter of the plug element is orientated towards the atrium, while the lower perimeter is orientated towards the ventricle.

The diameter of the frusto-conical plug at its upper perimeter (Dᵤ) and the diameter thereof at its lower perimeter (Dₗ) are selected such that the diameter of the cardiac valve annulus into which the device will be implanted (Dₐ) is smaller than Dᵤ and greater than Dₗ.

Preferably Dᵤ is in the range of 50 to 35 mm and Dₗ is in the range of 20 to 35 mm. In one preferred embodiment, Dᵤ is 40 mm and Dₗ is 30 mm

In one particularly preferred embodiment, the plug element has the form of an inverse, hollow frusto-conical plug having a central lumen and sides that taper inwards from above to below. In most embodiments, the tapering sides comprise a biocompatible fabric, such as PET or Dacron, or mixtures thereof.

In some embodiments, the tapering sides of the plug element are supported and/or created by the presence of plug support elements, which are lateral extensions arising from the body of the intracardiac device, wherein each of lateral extensions is disposed at a greater distance from the device body at its upper extremity than at its lower extremity. In these embodiments, the tapering fabric sides of the plug element are sutured to said plug support elements.

When used in conjunction with the prosthetic valve and valve support devices of the present invention, the aforementioned plug support elements are generally attached at their medial ends to the tubular stent-like portion of the device, by means of surgical suture material.

In other embodiments, the aforementioned biocompatible fabric sides of the plug are not supported by plug support elements, but rather are sutured or otherwise secured to other metallic portions of the intracardiac device. In one preferred embodiment of this type, the intracardiac device is a prosthetic cardiac valve or valve support device as disclosed herein, wherein the fabric sides of the plug are sutured to the anchoring elements, the tubular stent-like element and/or (when present in the cardiac valve embodiment), the cylindrical lower sleeve support stent (as will be described in more detail hereinbelow).

In some preferred embodiments of this aspect of the invention, the plug has the form of a single continuous frusto-conical element. In other preferred embodiments, the plug element is not a single continuous element, but rather consists of one or more discrete frusto-conical segments, as will be described in more detail hereinbelow.

Thus, in one aspect, the present invention is directed to an intracardiac device for implantation at a cardiac valve annulus selected from the group consisting of a prosthetic atrioventricular valve as disclosed hereinabove and a cardiac valve support device as disclosed hereinabove, wherein said intracardiac device comprises a hollow plug element for improving sealing of the device within a native cardiac valve annulus, wherein said plug element is frusto-conical with upper and lower perimeters having generally circular outlines, wherein the diameter at the upper perimeter is larger than the diameter at the lower perimeter, and wherein the diameter of the cardiac valve annulus into which said intracardiac device is to be implanted is smaller than the diameter of said hollow plug at its upper perimeter and larger than the diameter at its lower perimeter.

In one preferred embodiment of this aspect of the invention, the hollow plug element comprises one or more strips of biocompatible fabric that are wound horizontally, either completely or partially, around the external surface of the tubular stent-like portion and/or to the anchoring elements of said device, wherein said one or more strips are attached to said stent-like portion and/or to said anchoring elements, and wherein said fabric strip is orientated such that its upper border is disposed both above and lateral to its lower border.

In some preferred embodiments, the intracardiac device further comprising a plurality of plug support elements attached to or arising from the tubular stent-like portion of the device, wherein said plug support elements are disposed such that they extend outwards from the outer surface of said tubular stent-like portion, thereby providing a sloped attachment surface to which the fabric strip may be attached to said tubular stent-like portion. The plug support elements may have any suitable and convenient shape, including, but not limited to straight arms, curved arms and inverted ‘V’ shaped arms.

In use, the presence of the frusto-conical plug element permits the intracardiac device (such as the prosthetic valve and valve support devices disclosed herein) to which it is attached to be inserted into a cardiac valve annulus (preferably an atrio-ventricular annulus, more preferably the mitral annulus) form its upper (i.e. atrial) aspect and then manipulated such that the device moves downwards until the plug element becomes snuggly seated within the annulus. This occurs when the diameter of the portion of the frusto-conical sidewalls of the plug element that is in contact with the soft tissue of the annulus has the same or slightly larger outer diameter than the diameter of said annulus. By way of analogy, the plug element functions in a similar manner to a frusto-conical bung when inserted into the open neck of a vessel such as a glass test-tube or conical flask. After the bung has been inserted to the greatest possible depth (limited by frictional resistance between said bung and the vessel wall), it is neither possible to move the bung further down into the vessel, nor to withdraw said bung in an upward direction without the application of considerable force.

It will therefore be appreciated that the incorporation of a frusto-conical plug element into the structure of an intracardiac device (including, but not limited to, the prosthetic valve and valve support devices of the present invention) improves both the stability of said device within the mitral (or other cardiac) annulus, and prevents (or reduces to the greatest possible extent), leakage between the outer wall of said device and said mitral annulus.

For the sake of clarity, it should be noted that while the term “frusto-conical” is used to define the shape of the plug element, this element may also be conceptualized or defined in different ways. In essence, the plug element is a fabric strip that is wound around all or part of the outer circumference of the tubular stent-like portion (and/or to plug support elements attached to said stent-like portion), in such a way that said strip is constructed such that its upper border (or perimeter) is longer than its lower border. Furthermore, this fabric strip is connected to the external surface of the intracardiac device such that the upper border thereof is disposed both above and lateral to the lower border. In this way, the fabric strip presents a sloping surface (i.e. sloping from above to below in a medial direction), similar to the sidewalls of a frusto-conical plug.

The device of the present invention will now be described in more detail, with reference to the following accompanying figures:

### BRIEF DESCRIPTION OF THE FIGURES

FIG. 1 is a lateral view of one preferred embodiment of the prosthetic replacement valve of the present invention that is designed to be fitted with two valve leaflets. (Leaflets and fabric cover not shown.)

FIG. 2 is a side view of the same embodiment shown in FIG. 1.

FIG. 3 is a photographic plan view, taken from above, of a preferred embodiment of the present invention having two valve leaflets, following the attachment of a fabric cover to the metallic framework of the device.

FIG. 4 is a side view of the same embodiment shown in FIG. 3.

FIG. 5 is a side view of one preferred embodiment of the prosthetic replacement valve of the present invention that is designed to be fitted with three valve leaflets. (Leaflets and fabric cover not shown.)

FIG. 6 is a photographic plan view, taken from below, of a preferred embodiment of the present invention having three valve leaflets, following the attachment of a fabric cover and pericardial leaflets to the metallic framework of the device.

FIG. 7 depicts, in side view, one embodiment of the invention in which the device is fitted with a sleeve support frame in the form of a full stent.

FIG. 8 shows, in side view, an embodiment of the invention in which the sleeve support frame is constructed in the form of a half stent.

FIG. 9 presents a side view of a device of the present invention, wherein the sleeve support frame is provided by a single wire.

FIG. 10 depicts, in side view, and embodiment of the present invention in which the sleeve support frame comprises a series of low profile posts.

FIG. 11 provides a view from above of a device of the present invention comprising a plurality of plug support elements attached to the lower margin of the tubular stent-like portion of said device.

FIG. 12 presents a side view of the embodiment depicted in FIG. 11.

FIG. 13 provides an enlarged view of the plug support elements depicted in FIGS. 11 and 12.

FIG. 14 provides a view from above of a device of the present invention comprising a plurality of plug support elements attached to the upper margin of the tubular stent-like portion of said device.

FIG. 15 presents a side view of the embodiment depicted in FIG. 14.

FIG. 16 provides an enlarged view of the plug support elements depicted in FIGS. 14 and 15.

FIG. 17 provides a view from above of an alternative embodiment of a device of the present invention comprising a plurality of plug support elements attached to the lower margin of the tubular stent-like portion of said device.

FIG. 18 presents a side view of the embodiment depicted in FIG. 17.

FIG. 19 provides an enlarged view of the plug support elements depicted in FIGS. 17 and 18.

FIG. 20 depicts, in side view, an intracardiac device of the present invention comprising a full circular fabric plug element attached to the lower portion of the tubular stent-like portion of said device.

FIG. 21 is a photographic representation, from below, of a device of the present invention, comprising two discrete frusto-conical plug elements attached to the anchoring elements and lower extension stent of said device.

FIG. 22 provides a side view of the same embodiment as depicted in FIG. 21.

FIG. 23 schematically depicts a device of the present invention comprising a plurality of plug support elements, following the implantation of said device within the mitral valve annulus.

FIG. 24 schematically depicts the successive stages involved in the transseptal delivery of a prosthetic valve of the present invention at the mitral annulus.

### DETAILED DESCRIPTION OF PREFERRED EMBODIMENTS

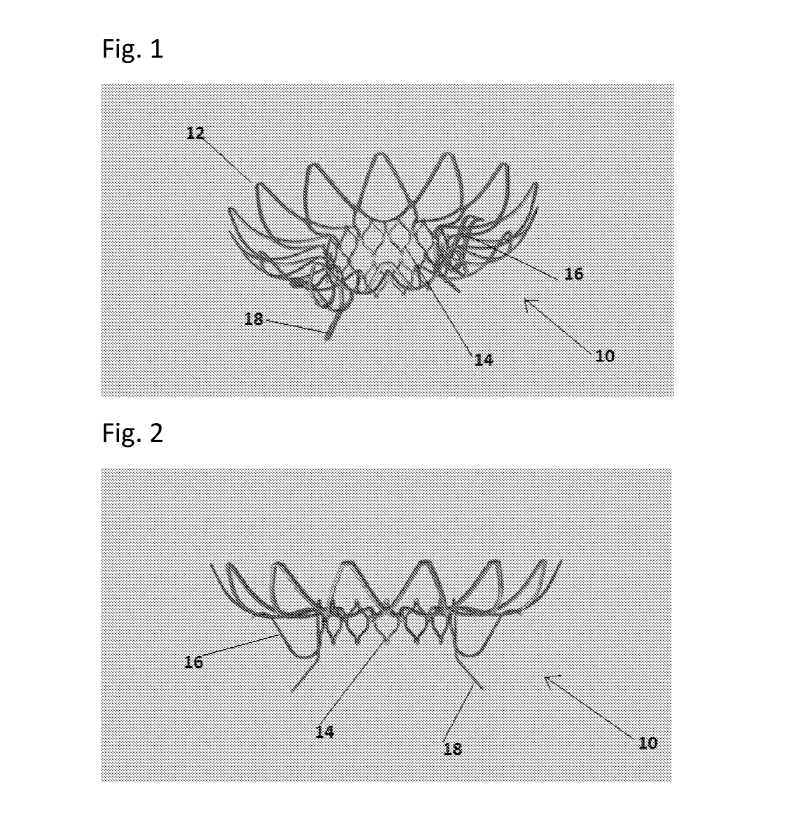

One typical embodiment of the replacement cardiac valve of the present invention is depicted, in lateral view, in FIG. 1. It may be seen that the device 10 comprises, in its outer and upper part a lateral crown-like portion 12, constructed in the form of a lattice. In the specific embodiment shown here, each cell of the lattice is approximately rhomboid. However, the lateral crown may be constructed in many different forms, having different sized and shaped lattice cells from those depicted here. Generally, as shown in this figure, the lateral crown portion 12 is shaped such that its upper portion curves upwards and laterally away from its lower region.

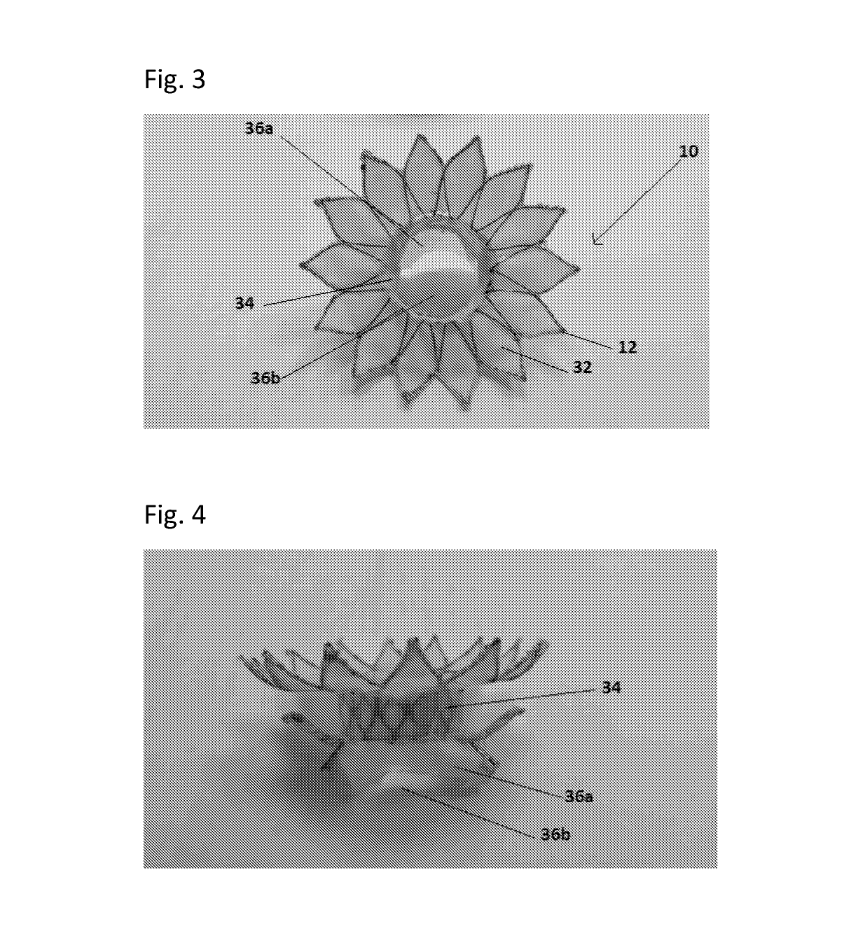

In its lower part, the lateral crown 12 is approximately horizontal (i.e. approximately at right angles to the longitudinal axis of the heart, when implanted in a cardiac annulus). This part of the lateral crown is continuous with the stent-like portion 14, which passes vertically downwards. When viewed from above (as shown in FIG. 3), the stent-like portion is generally circular in outline.

As in the case of the lateral crown, the stent-like portion in this particular embodiment is constructed of latticework having approximately rhomboid cells. This is only one particular form, provided for exemplary purposes—many other shapes are also possible and all of them fall within the scope of this disclosure. It is clear from FIG. 1, however, that this type of structure is ideally suited for the device of the present invention which needs to be capable of being radially compressed (crimped) into a delivery device, and then radially expanded upon deployment at the cardiac annulus.

Device 10 is also fitted with two anchoring arms (wings) 16 continuous with the stent-like portion at their medial ends. In this specific embodiment, the two arms are spaced approximately 180 degrees apart, such that the device may be implanted with said arms orientated at the commissures of the anatomical heart valve. In this way, it is possible to implant the replacement valve without interfering with the function of the native valve leaflets. Following implementation of the replacement valve, the native leaflets continue to open and close similar to before, except for the fact that instead of meeting at the native leaflet commissure during closure, the leaflets close around the replacement valve leaflet (or inferior sleeve, if present), thereby contributing to sealing and PVL prevention. In other embodiments, the replacement valve may comprise more than two anchoring arms.

The device shown in FIG. 1 further comprises a pair of commissural struts 18, which are continuous at their upper, medial ends with the stent-like portion 14. In the specific embodiment shown in this figure, the upper, medial root of each strut 18 is located close to and above the root of anchoring arm 16. Other spatial arrangements of the commissural struts relative to the anchoring arms are also possible, including specifically a direct connection between them, and all of them are included within the scope of the present invention.

FIG. 2 presents a side view of the same embodiment of the device 10 of the present invention that was shown in FIG. 1. In this figure, the vertically disposed tubular outline form of the stent-like portion 14 may be more clearly seen. This figure also illustrates the configuration of the anchoring arms 16, which are shaped such that they pass initially downwards from stent-like portion 14, and then turn upwards and laterally, in order that they may be used to contact the ventricular wall of the heart onto which they are implanted, and thereby provide mechanical support for device 10.

In the embodiment shown in this figure, the commissural struts 18 are shaped such that their upper portion passed vertically downwards from stent-like portion 14. At a certain point, the lower portion of each strut is angled laterally, such that it may be used to support a commissure of the fabric (or tissue) valve leaflet (shown in FIGS. 3 and 4). In other embodiments of this device the commissural struts may continue direct downwards (without the lateral angle), and there may be more than two such struts (corresponding to more than two leaflets).

While only the metallic elements of the device are shown in the embodiment depicted in FIGS. 1 and 2, the next two figures, FIGS. 3 and 4, are photographic representations of similar embodiments following the attachment of a fabric cover and leaflets. Thus, in FIG. 3, which is a plan view of device 10 taken from above, the outline shape of lateral crown portion 12, together with fabric crown cover 32, is clearly seen. Also shown are two valve leaflets 36a and 36b, constructed from a biocompatible fabric that are attached to (and are continuous with) the upper fabric cover 34 that is itself attached to the stent-like region of the device. In another embodiment of this device (not shown here), the said leaflets may be constructed from biological tissue, for example pericardium.

FIG. 4, which is a side view of the same embodiment presented in FIG. 3, illustrates the arrangement of the valve leaflets, 36a and 36b as inferior free extensions of the fabric cover 34 that is attached to the stent-like portion of the device.

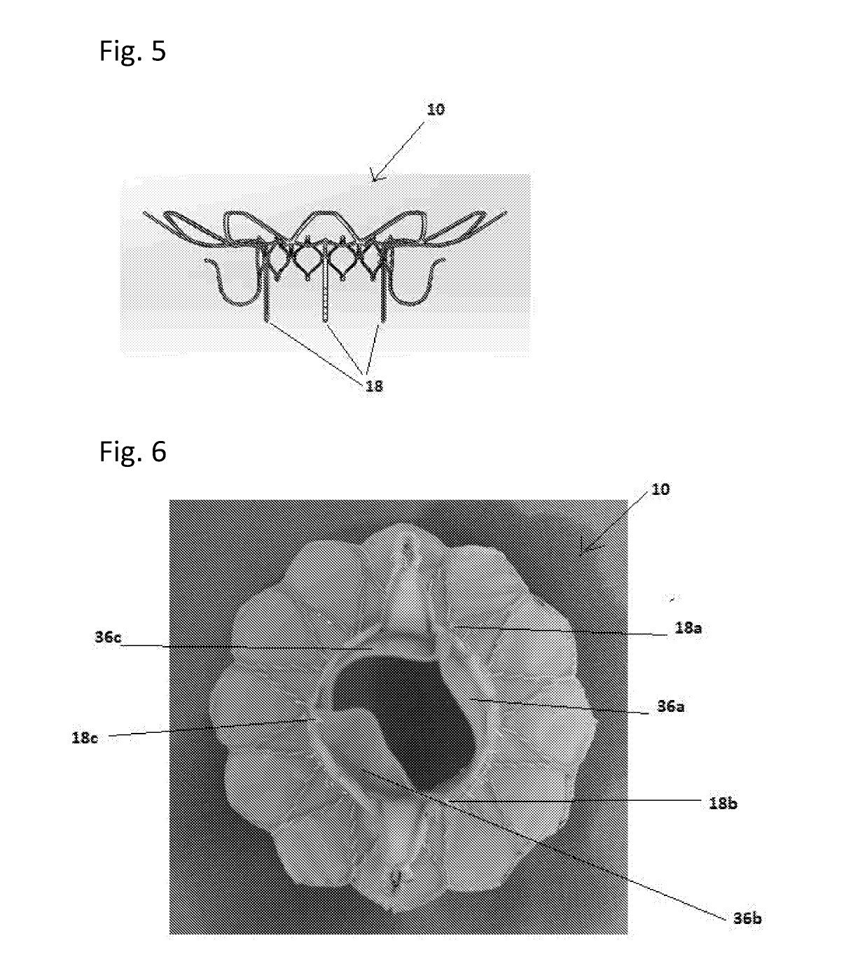

FIG. 5 presents a side view of another preferred embodiment of the prosthetic replacement valve of the present invention that is designed to be fitted with three valve leaflets. (The leaflets themselves and the fabric cover are not shown for clarity.) It will be noted that this embodiment of the device 10 is similar to that shown in FIG. 2, with the exception that it has three commissural struts 18 (instead of two as in the two-leaflet embodiment). Furthermore, the struts used in this specific embodiment are straight rather than angled as shown in FIG. 2.

The same embodiment as depicted in FIG. 5 is also shown in FIG. 6, but this time following the attachment of the fabric covering and the valve leaflets. In this plan view (from below) it may be seen that device 10 comprises, in this embodiment, three valve leaflets, 36a, 36b and 36c. In addition to being attached via their superior sides to the stent-like portion of the device, the leaflets are attached via their lateral sides to the commissural struts 18a, 18b and 18c.

As explained hereinabove, in some particularly preferred embodiments of the present invention, the replacement valve comprises a lower (inferior) sleeve that is attached superiorly to the lateral crown, the stent-like portion or the boundary region between those two regions of the device. The sleeve—which may be constructed from a biocompatible fabric, a biological tissue (preferably pericardium) or mixtures thereof—may be attached by suturing either directly to the metallic frame of the device (at one of the locations mentioned above) or to the fabric or pericardial cover that is attached to said frame. The essentially cylindrical sleeve is disposed such that is aligned with the longitudinal axis of the device (and therefore, the same axis of the heart following deployment), with a lower free margin, wherein said sleeve surrounds and partially or fully encloses the valve leaflets from their external aspect. In this way, the lower sleeve is ‘sandwiched’ between two sets of leaflets: the native valve leaflets which continue to open and close against the outer surface of the sleeve, and the replacement valve leaflets which open within the central cavity of the sleeve and whose fully open position is defined and/or limited by inner wall of said sleeve.

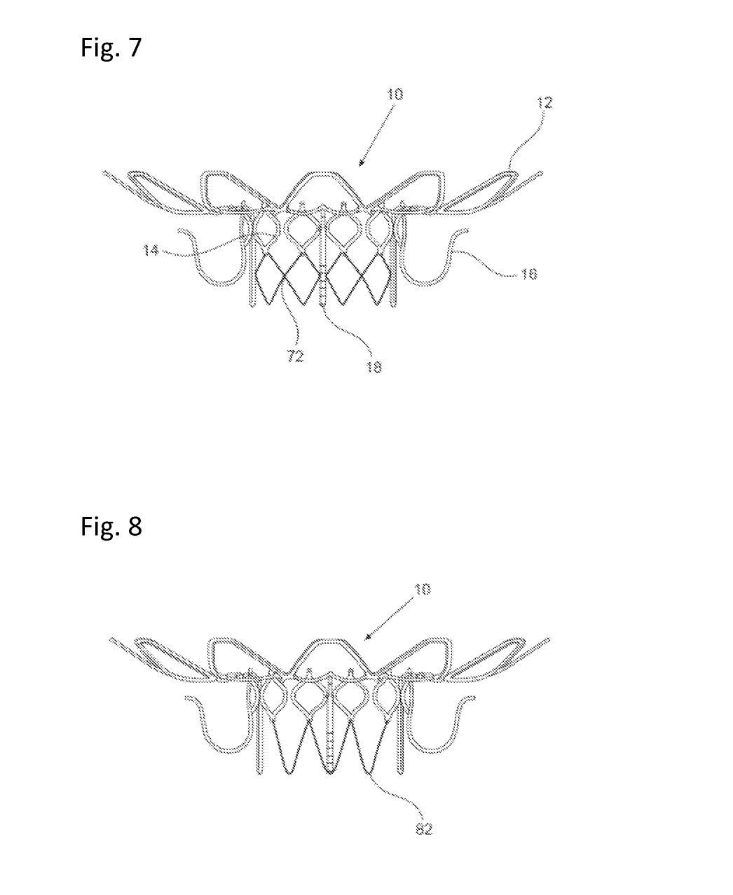

In some embodiments of the invention, the lower sleeve is unsupported, being attached at its upper end only, as described above. In most preferred embodiments, however, the sleeve is supported by (and sutured to) a metallic sleeve frame, in order to provide additional support therefor. Many different types of sleeve support frame may be used. FIG. 7 depicts a device of the present invention 10 fitted with a sleeve support that is in the form of a full cylindrical stent 72 that is attached in its upper portion to the stent-like region 14 of the device. This figure also clearly shows the commissural struts 18, the anchoring arms 16 and the lateral crown portion 12.

In another preferred embodiment, as shown in FIG. 8, device 10 contains a sleeve support frame that is constructed in the form of a half-stent 82.

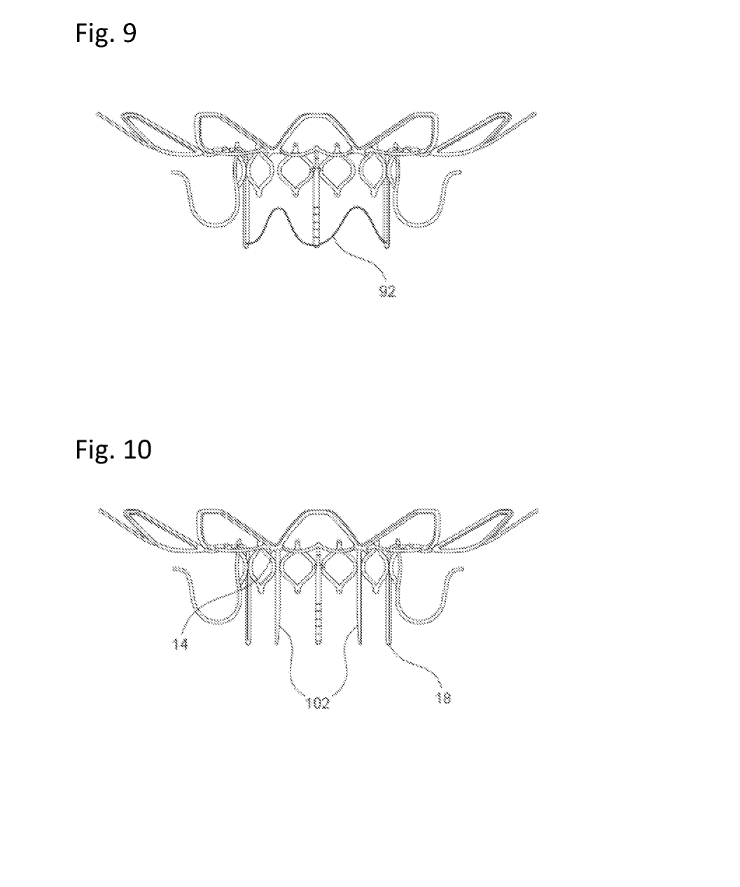

Simpler support frames, constructed from elements that occupy less space are shown in FIGS. 9 and 10. Thus, FIG. 9 presents an embodiment in which the sleeve support frame 92 is in the form of a wire 92 that provides direct support for the lower portion of the sleeve that is attached thereto. In contrast, in the embodiment shown in FIG. 10, the sleeve support frame is supplied by a plurality of low profile posts 102 attached at their upper ends to the stent-like portion of the device 14, and arranged parallel with the commissural struts 18.

The various embodiments of the sleeve support frame shown in FIGS. 7-10 and described above are provided for the purposes of illustration only, and do not limit the present invention in any way. Many other forms of support sleeve may also be contemplated, and all of these forms will fall within the scope of the present invention.

As disclosed hereinabove, the present invention also encompasses additional means for reducing leakage between the implanted intracardiac device (e.g. a prosthetic valve or valve support device of the present invention, or any other type of intracardiac device), and the anatomical valve annulus. These means primarily comprise a frusto-conical shaped plug element, constructed from a biocompatible fabric (for example Dacron or PTE). The attachment of this plug element to the metallic framework of the device is generally achieved by means of surgical sutures. As described above, in some embodiments of this aspect of the invention, the device may further comprise a plurality of plug support elements, whose shape and disposition within the device facilitate the creating and/or attachment of the frusto-conical plug element.

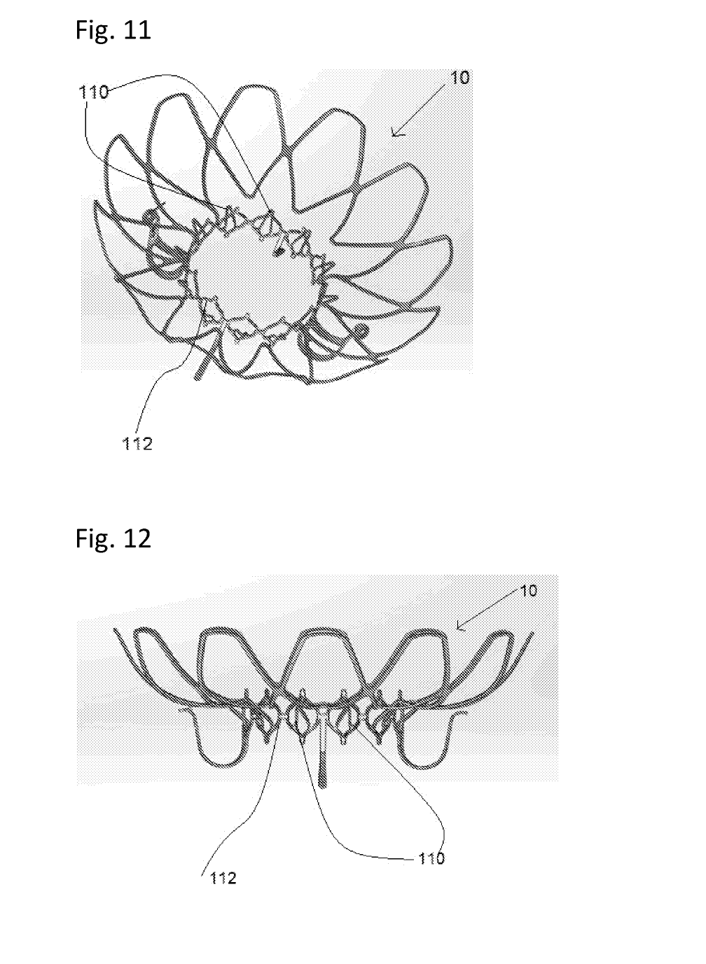

FIGS. 11 and 12 show, respectively, an upper perspective view and a side view of a further preferred embodiment of an intracardiac device of the present invention 10, wherein said device comprises a plurality of plug support elements 110. Each of said plug support elements has the form of an inverted letter ‘V’, and is attached by its two free ends to the lower margin of the tubular stent-like element 112. It will be noticed from both of these figures (and especially from FIG. 11) that each plug support element 110 is angled upwards and laterally, such that an imaginary line drawn such that it would connect the apices of each ‘V’-shaped plug support element would describe a circle having a larger diameter than the diameter of tubular stent-like element 112. Although not shown in these two figures, during the manufacture of device 10, a length of PET or Dacron (or similar biocompatible) fabric is sutured to the outer surface of each of the plug support elements, thereby forming a frusto-conical plug element having an upper perimeter and diameter that is greater than its lower perimeter and diameter, respectively. As explained hereinabove, during use, said frusto-conical plug element enables a closer fit between the device and the mitral valve annulus during and after implantation.

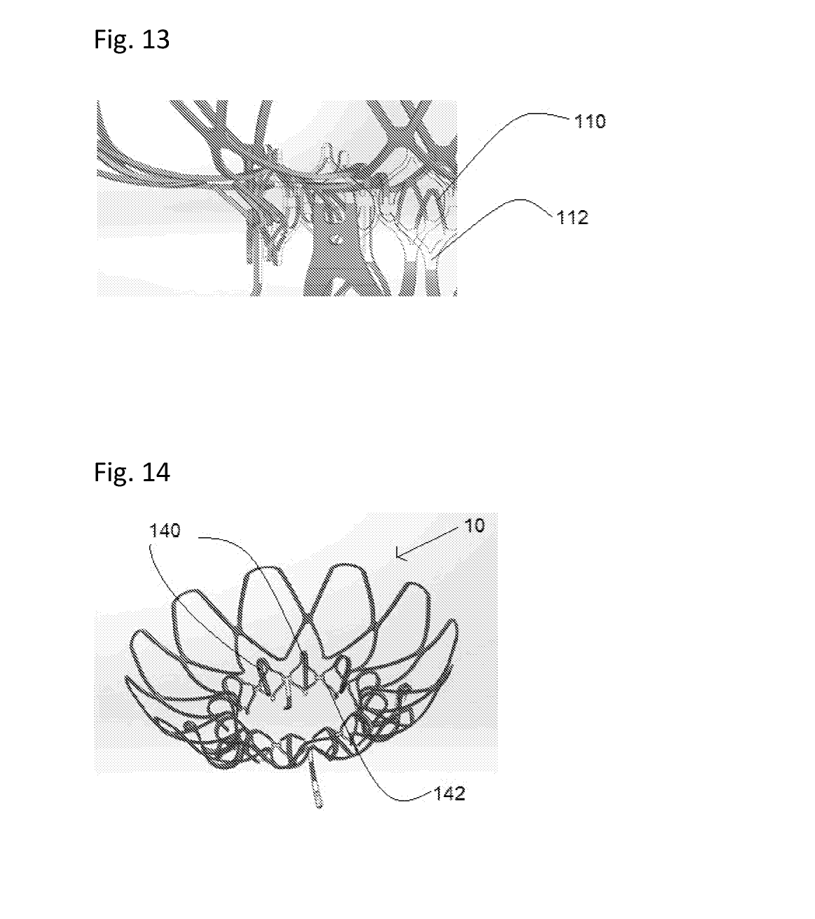

The attachment of the plug support elements 110 to the tubular stent-like element 112 is shown more clearly in the enlarged view presented in FIG. 13.

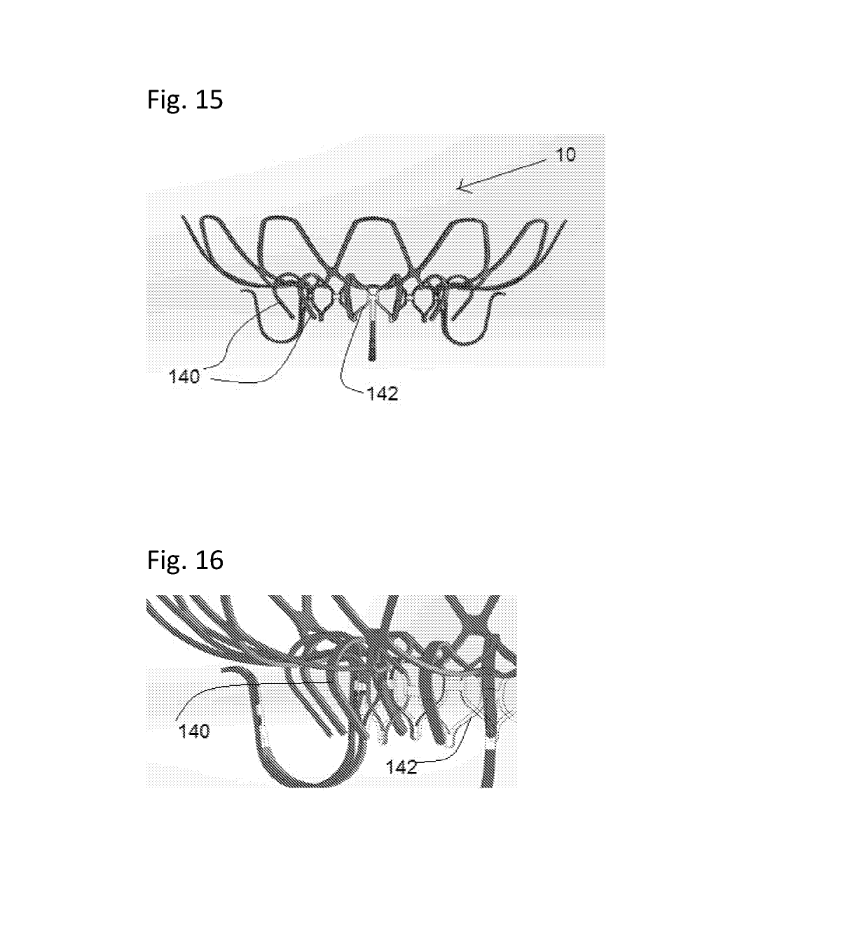

FIGS. 14 and 15 show, respectively, an upper perspective view and a side view of another preferred embodiment of an intracardiac device of the present invention 10, wherein said device comprises a plurality of plug support elements 140. However, said plug support elements in this embodiment differ from those depicted in FIGS. 11-13, both with regard to their shape and with regard to their attachment to the device. Thus, in the presently-described embodiment the plug support elements 140, have the form of curved narrow arms which are attached to the upper margin of the tubular stent-like element 142. The curvature of the plug support elements—which is initially slightly upwards and laterally from their attachment point on tubular stent-like element 142, and then downwards and medially—is seen more clearly in the enlarged view shown in FIG. 16. As in the case of the previous embodiment (shown in FIGS. 11-13), the fabric cover attached to the outer surfaces of plug support elements 140 provides the lateral surface of the frusto-conical plug element.

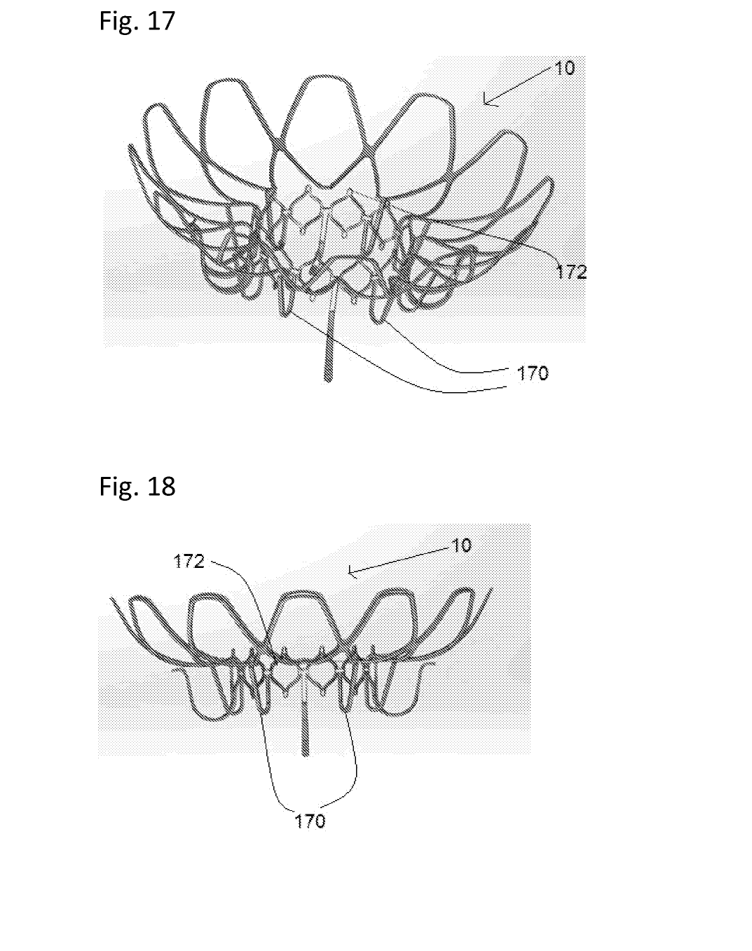

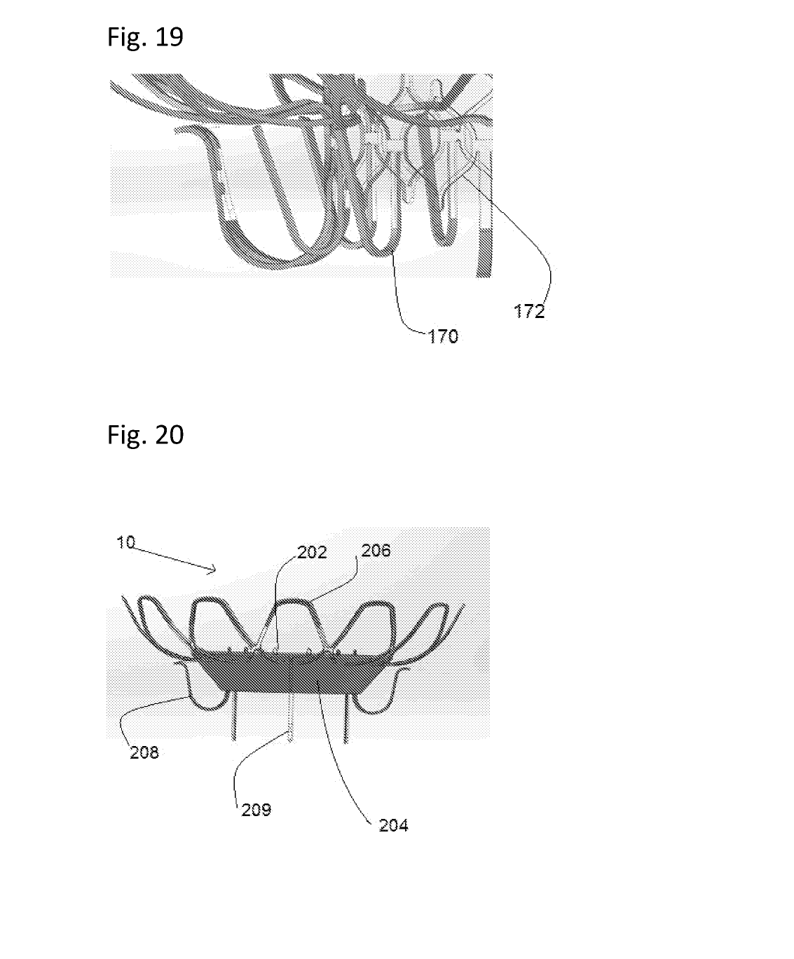

FIGS. 17 and 18 show, respectively, an upper perspective view and a side view of yet another preferred embodiment of an intracardiac device of the present invention 10, wherein said device comprises a plurality of plug support elements 170. In this embodiment, said plug support elements are narrow curved arms (as in the case of the embodiment of FIGS. 14-16). However, in this case, the plug support elements 170 are attached to the lower margin of the tubular stent-like element 172. Furthermore, as shown more clearly in the enlarged view presented in FIG. 19, the curvature of the plug support elements is opposite to that in the previously described embodiment.

As explained hereinabove, the fabric cover which creates the side wall of the frusto-conical plug element may form a complete circle, yielding a single, completely circular plug. Alternatively, several discrete fabric covers which are unconnected to each other may be employed, such that the resulting plug element is divided into a number of separate segments.

FIG. 20 provides a side view of device 10 of the present invention in which a single, intact frusto-conical plug is created by attaching a single length of biocompatible fabric 204 all the way around the circumference of the tubular stent-like portion 202 (of which only the upper extremities are seen in this figure, the rest of said stent-like portion being obscured by the fabric). As shown in this figure, device 10 further comprises three commissural posts 209 and two anchoring elements 208 attached to stent-like portion 202. It is to be noted that in this embodiment, the frusto-conical plug formed by the biocompatible fabric 204 is located beneath and on the medial side of lateral crown portion 206. Finally, although not visible in FIG. 20, in this embodiment, device 10 does not contain any plug support elements, the biocompatible fabric portion 204 being preformed into a frusto-conical envelope shape and attached directly to stent-like portion 202. In some specific embodiments of this type, the preformed frusto-conical shape is achieved by means of incorporating one or more Nitinol wire formers into the biocompatible fabric cover.

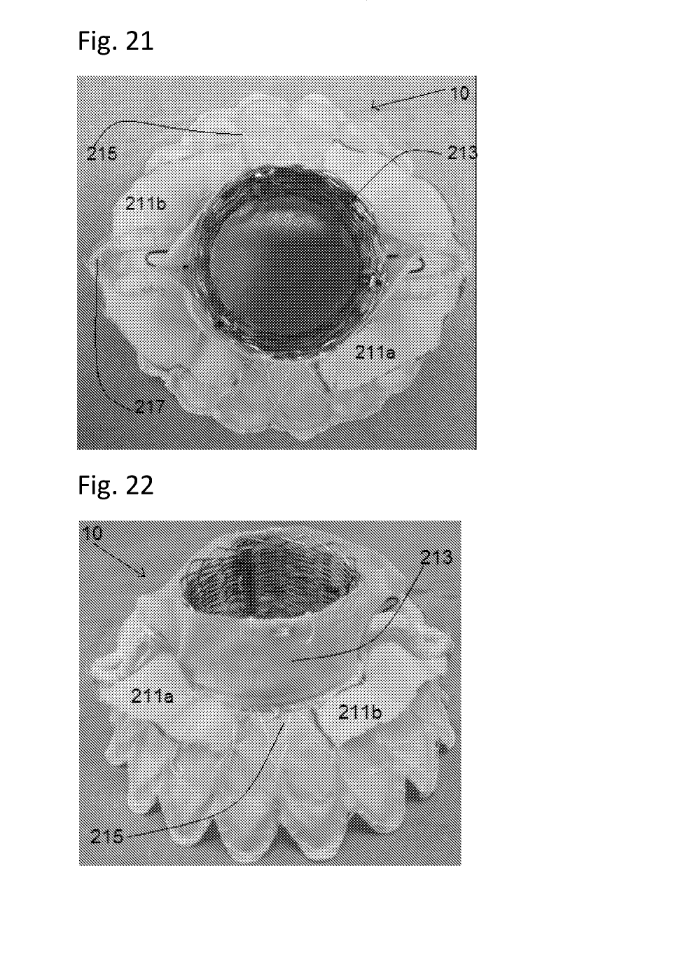

FIGS. 21 and 22 present, respectively, a lower view and side perspective view of a different embodiment of a device 10 of the present invention. In this embodiment, the device comprises two anchoring elements 217 and a fabric-covered lower extension stent 213 (the fabric cover of which obscures the tubular stent-like portion in these figures). The plug element in this embodiment comprises two discrete sections 211a and 211b, said plug element comprising two separate biocompatible fabric covers which are sutured to both anchoring elements 217 and to the tubular stent-like portion. As shown in FIG. 22, both sections of the plug element are located on the inferior side (i.e. ventricular face) of laterally-expanded crown portion 215.

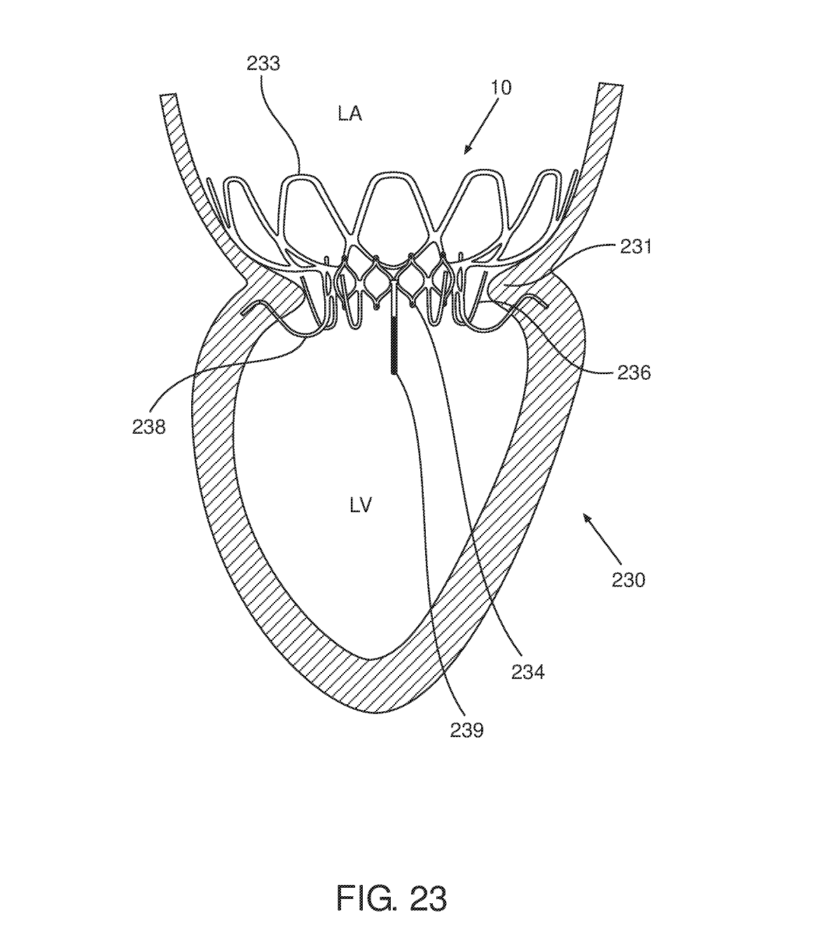

As explained hereinabove, the purpose of the plug element is to enable better seating of the intracardiac device (e.g. the prosthetic mitral valve and mitral valve support devices of the present invention) in the mitral annulus, thereby reducing or preventing leakage between the irregularly shaped soft tissue of the annulus and the device itself. FIG. 23 shows an embodiment of the device essentially the same as that depicted in FIGS. 17-19 and described hereinabove, wherein device 10 has been brought into its expanded working configuration at the mitral valve annulus 231. As seen in this figure, the upper laterally-expanded crown portion 233 is located in the lower portion of the left atrium LA, and in contact with the atrial wall. Two anchoring elements 238 are disposed within the upper portion of the left ventricle LV, with their distal ends making contact with the ventricular wall beneath the annulus. The tubular stent-like portion 234 of the device is located at the level of the annulus, with the plug attachment elements 236 making contact with the annulus itself. Commissural posts 239 (only one shown in this figure) are also attached to the tubular stent-like portion. Although not shown in this figure (for the sake of clarity), in use, the entire device will be covered with a biocompatible fabric. In this way, the fabric cover around plug attachment elements 236 together with said plug attachment elements themselves will form a frusto-conical plug element, which when manipulated in a downward direction will allow the device to move slightly until the diameter of the portion of said plug element in contact with the annulus is the same, or slightly larger than, the diameter of the annulus itself. In this way, fluid leakage between the device and the annulus is significantly reduced or prevented.

The metallic framework of the device (i.e. the stent-like and lateral crown portions and the lower sleeve support frame) may be formed from either a sheet of a shape-memory metal (preferably Nitinol), or a tube of the same material. Generally, the metal used to manufacture the device (both in the case of tubular material or when initially in the form of a sheet) has a thickness in the range of 0.2-1.5 mm.

When the device is constructed from sheet metal, said sheet is formed around a mandrel into a tube and welded along the join.

The open latticework form of the lateral crown and stent-like portions is created by means of removing unwanted portions of the metal by means of laser cutting, etching or any other standard method known to the skilled artisan in this field. In the case of devices formed from sheet metal, this stage of forming the lattice may be either performed prior to folding the sheet into a tube or subsequent thereto.

The laterally expanded form of the lateral crown is formed using an appropriately sized and shaped mandrel.

In some preferred embodiments, the anchoring arms and the commissural struts (when present) are formed from the same single tube or sheet of the shape memory metal. In other embodiments, these elongate elements are formed separately, from the same or similar material, and then attached to the stent-like portion of the device by, for example, laser welding.

As mentioned hereinabove, the metallic framework of the device of the present invention is covered with either an impermeable biocompatible polymeric fabric material (such as, for example, Dacron, polyurethane, polyamide, and so on) or with a biological tissue such as pericardial tissue of either animal or human origin. In some embodiments, the covering material may also comprise both Dacron (or other suitable polymer) and pericardial (or other) tissue. In such composite embodiments, the valve leaflet portion of the cover (i.e. the lower portion that is attached superiorly to the stent frame) is generally manufactured from pericardium, while the portions attached to the metallic framework may be manufactured entirely from fabric or from a mosaic of fabric and pericardial tissue. Similarly, both the lower sleeve and the valve leaflets may be constructed from any of the aforementioned materials or combinations thereof.

The upper cover, lower sleeve and valve leaflets (whether made from fabric or biological tissue or both) are generally attached to the metallic framework of the device by means of surgical sutures.

Generally, the external diameter of the stent-like portion when expanded to its maximum value (i.e. when no more expansion is possible because of the restraining influence of the fabric cover) is in the range of 22 to 36 mm.

The height of the stent-like portion of the device, when in its expanded, deployed configuration is generally in the range of 14 to 24 mm.

The overall diameter of the device, measured from one point on the outer margin of the lateral crown portion to a similar point located 180 degrees around the circumference of the device is preferably in the range of 40 to 80 mm.

The prosthetic atrioventricular valve of the present invention may be delivered by any suitable method known in the art including (but not limited to) open-heart approaches, minimally invasive transapical approaches, as well as transseptal and other transvascular routes of administration. However, in view of its unique design, the present invention is ideally suited to transseptal administration. The reason for this, as mentioned above, is that the device of the present invention, despite being a mitral valve fitted with all of the elements required for providing mechanical stability and fluid sealing, is capable of being radially crimped to a diameter of 24 FR, or even less (as opposed to approximately 32 Fr in the case of most if not all prior art devices). This advantage is achieved by the unique spatial arrangement of the three main regions of the device: the lateral-crown region, the stent-like portion and the sleeve support frame (where used). Thus, in contradistinction to most prior art prosthetic atrioventricular valves, when the present device, is crimped radially, the three aforementioned distinct regions become offset along the longitudinal axis. In this way, the bulky lateral crown region does not interfere with the crimping of the other elements, since it occupies its own ‘crimp space’ at the upper end of the device. Similarly, the mid region of the crimped device is occupied by the stent-like portion only, which in turn does not interfere with the crimping of the inferiorly placed lower sleeve support frame. It may thus be appreciated that the present device is, in a sense, ‘reversely telescopic’ since it becomes elongated, with longitudinal separation between each of the three sections when in its non-working, crimped delivery configuration. Upon implementation, however, once the device has been removed from the confines of the delivery catheter (or other delivery device), these three sections of the device, while expanding radially, also move toward each other, thereby reducing the overall longitudinal length of the device (compared to its crimped condition).

An additional feature of the present invention which contributes to its ability to radially crimp to a very small diameter is the fact that unlike many prior art devices, the presently-claimed device is fitted with anchoring elements (generally in the shape of arms or wings) which occupy a much smaller area and volume than the comparable elements commonly used in the past.

The fact that the presently claimed replacement valve may be crimped down to diameters of 24 Fr or less is highly significant, since it permits loading of the device into delivery catheters that are small enough to be used for transvascular delivery in a manner that is both effective and safe.

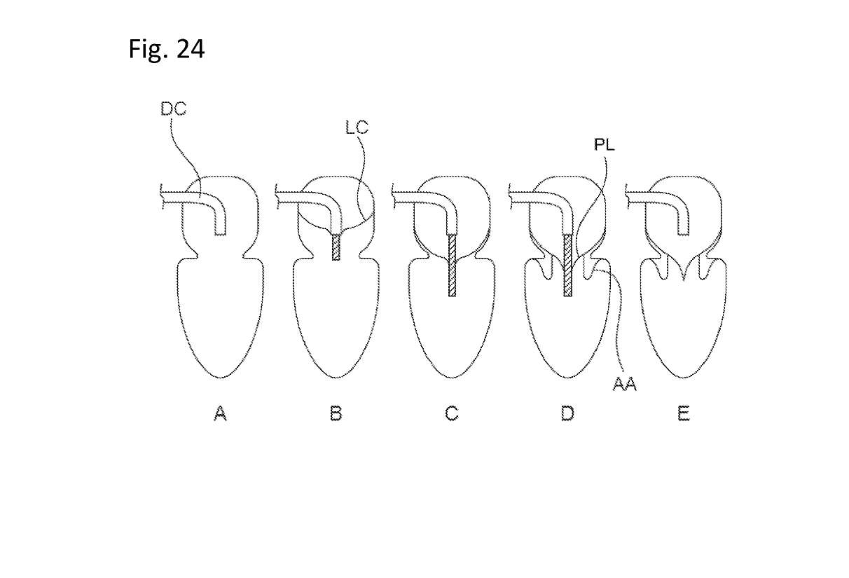

Many suitable transvascular delivery systems for transporting prosthetic cardiac valves (mainly the smaller-diameter aortic valves) through the vasculature are well known in the art. In general terms, the device of the present invention, when used to replace a mitral valve is crimped and loaded into a small diameter (approximately 24 Fr) delivery catheter (or other delivery conduit), in the manner indicated hereinabove. Then, following percutaneous entry into a blood vessel (generally in one of the limbs), the delivery catheter is steered in the vasculature until it enters the right atrium of the heart, via the superior or inferior vena cava. The next stages of the procedure are depicted schematically in FIG. 24, as follows:

A. The delivery catheter (DC) is advanced across the interatrial septum and enters the left atrium.

B. The lateral crown region (LC) of the device is then released from the delivery catheter and allowed to passively expand.

C. The position of the delivery catheter (or a specific portion thereof) is then adjusted such that the lateral crown is moved downwards, along the longitudinal axis of the heart, such that it obtains its working position (along the upper side of the mitral annulus and the lateral wall of the left atrium).

D. The anchoring arms (AA) are then permitted to spring open into their working position (making contact with the upper portion of the left ventricular wall). This may, in one particular embodiment, be achieved by releasing a control wire that has hereto restrained the anchoring arms in their crimped delivery conformation. It is to be noted that at this stage the prosthetic valve leaflets (PL) open and close in response to the changing hemodynamic pressure over the course of the cardiac cycle (since the procedure is performed on a beating heart). However, said prosthetic leaflets may not be able to achieve full closure at their commissural line(s), due to the presence of the inner conduit or rod or wires of the delivery system.

E. Finally, the delivery catheter is completely removed from the replacement valve, thereby enabling the prosthetic valve leaflets to open and close to their fullest extent.

The clinical methods used in the minimally invasive transvascular delivery of crimped or otherwise size-reduced intracardiac devices are well known to skilled artisans in this field. Further details may also be obtained from many different published sources, including U.S. Pat. No. 7,753,923 and WO 2008/070797.

The prosthetic atrioventricular valve and atrioventricular valve support of the present invention possess inter alia the following advantages when compared with various other intracardiac devices of the prior art:

1. The device can be crimped down and inserted into approximately 24 Fr (or less than 32 Fr) diameter delivery conduit. It is therefore possible to safely deliver a bulky mitral valve, without causing trauma to the vasculature or other tissues, via a transseptal route. In addition to the safety aspect, the very small crimp diameter permits the device, in its delivery conformation to be easily maneuvered around tight angles in the vasculature during the delivery procedure.

2. The prosthetic valve of the present invention is generally fitted with minimalistic anchoring (or attachment) elements (thin wings or arms) that may contact with the ventricular tissue only at two discrete points. This is clearly advantageous in relation to prior art devices in which there usually is 360-degree attachment to the native valve annulus, in order to achieve the required mechanical stability, or more prominent (and thicker) mechanisms to capture the native leaflets or the basis of the leaflet (e.g. anatomical trigone). Furthermore, since the two discrete anchoring points in the present invention are generally located along the commissural line of the native mitral valve, the native leaflets are able to continue to work even after full implementation of the replacement valve. In this way, the native leaflets are able to contribute to fluid sealing and prevention of paravalvular leakage. This contrasts with the situation in most prior art systems, in which the replacement valve is attached to the native leaflets, thereby preventing this advantageous use.

3. Very short intracardiac procedure time (2-10 minutes), with delivery and implantation performed on a beating heart.

4. Ease of manufacture (from Nitinol tube).

1. A prosthetic atrioventricular valve, suitable for transvascular delivery to a heart valve annulus, comprising a tubular stent-like portion that is continuous in its upper part with a laterally expanded crown-like portion, wherein at least said stent-like portion is covered with a covering layer comprising a material selected from the group consisting of a biocompatible fabric, pericardial tissue and a combination of both a fabric and pericardial tissue, and wherein said stent-like portion is fitted with two or more valve leaflets.

2. The prosthetic valve according to claim 1, further comprising a tubular lower sleeve surrounding the two or more valve leaflets.

3. The prosthetic valve according to claim 2, wherein the lower sleeve is attached to a lower support frame, wherein said frame is selected from the group consisting of a full stent, half stent, single wire support and support posts.

4. The prosthetic valve according to claim 1, further comprising one or more anchoring arms, wherein said anchoring arms each have a root that arises from the stent-like portion of the device.

5. The prosthetic valve according to claim 1, further comprising one or more commissural struts arising from and continuous with the stent-like portion of said device.

6. The prosthetic valve according to claim 1, wherein said valve is fitted with three valve leaflets only.

7. The prosthetic valve according to claim 1, wherein said valve is fitted with two valve leaflets only.

8. A prosthetic atrioventricular valve, suitable for transvascular delivery to a heart valve annulus, comprising a tubular stent-like portion that is continuous in its upper part with a laterally expanded crown-like portion, and in its lower portion with a lower sleeve support frame, wherein said stent-like and crown like portions and said sleeve support frame are covered with a covering layer comprising a material selected from the group consisting of a biocompatible fabric, pericardial tissue and a combination of both a fabric and pericardial tissue, and wherein said stent-like portion is fitted with two or more valve leaflets and two or more anchoring arms.

9. The prosthetic valve according to claim 8, wherein the lateral crown-like portion, the tubular stent-like portion and the lower sleeve support frame are arranged such that upon crimping said device, each of said portions become offset from each other along the longitudinal axis of the crimped valve.

10. A cardiac valve support device suitable in size, shape and construction for transvascular delivery and implantation at an atrioventricular annulus, comprising a tubular stent-like portion that is continuous, in its upper part with a laterally expanded crown-like portion, wherein at least said stent-like portion is covered with a covering layer comprising a material selected from the group consisting of a biocompatible fabric, pericardial tissue and a combination of both a fabric and pericardial tissue.

11. The valve support device according to claim 10, further comprising a tubular lower sleeve.

12. The valve support device according to claim 11, wherein the lower sleeve is attached to a lower support frame, wherein said frame is selected from the group consisting of a full stent, half stent, single wire support and support posts.

13. The valve support device according to claim 10, further comprising one or more anchoring arms, wherein said anchoring arms each have a root that arises from the stent-like portion of the device.

14. The valve support device according to claim 10, further comprising one or more commissural struts arising from and continuous with the stent-like portion of said device.

15. An intracardiac device for implantation at a cardiac valve annulus selected from the group consisting of
a prosthetic atrioventricular valve comprising a tubular stent-like portion that is continuous in its upper part with a laterally expanded crown-like portion, wherein at least said stent-like portion is covered with a covering layer comprising a material selected from the group consisting of a biocompatible fabric, pericardial tissue and a combination of both a fabric and pericardial tissue, and wherein said stent-like portion is fitted with two or more valve leaflets, and
a cardiac valve support device according to claim 10,
wherein said intracardiac device comprises a hollow plug element for improving sealing of the device within a native cardiac valve annulus, wherein said plug element is frusto-conical with upper and lower perimeters having generally circular outlines, wherein the diameter at the upper perimeter is larger than the diameter at the lower perimeter, and wherein the diameter of the cardiac valve annulus into which said intracardiac device is to be implanted is smaller than the diameter of said hollow plug at its upper perimeter and larger than the diameter at its lower perimeter.

16. The intracardiac device according to claim 15, wherein the hollow plug element comprises one or more strips of biocompatible fabric that are wound horizontally, either completely or partially, around the external surface of the tubular stent-like portion and/or to the anchoring elements of said device, wherein said one or more strips are attached to said stent-like portion and/or to said anchoring elements, and wherein said fabric strip is orientated such that its upper border is disposed both above and lateral to its lower border.

17. The intracardiac device according to claim 16, further comprising a plurality of plug support elements attached to or arising from the tubular stent-like portion of the device, wherein said plug support elements are disposed such that they extend outwards from the outer surface of said tubular stent-like portion, thereby providing a sloped attachment surface to which the fabric strip may be attached to said tubular stent-like portion.

18. The intracardiac device according to claim 17, wherein the plug support elements are selected from the group consisting of straight arms, curved arms and inverted ‘V shaped arms.
